(19) United States
(12) Patent Application Publication
(10) Pub. No.: US 2025/0143716 A1
(43) Pub. Date: May 8, 2025

(54) DEVICES, SYSTEMS AND METHODS FOR VESSEL LIGATION

(71) Applicant: University of Pittsburgh - Of the Commonwealth System of Higher Education, Pittsburgh, PA (US)

(72) Inventors: Jason Y. Lee, Santa Clara, CA (US); Cyrus J. Darvish, Pittsburgh, PA (US); Timothy K. Chung, Pittsburgh, PA (US)

(21) Appl. No.: 18/937,726

(22) Filed: Nov. 5, 2024

Related U.S. Application Data
(60) Provisional application No. 63/596,463, filed on Nov. 6, 2023.

(51) Int. Cl. A61B 17/122; A61B 17/00; A61B 17/32; A61B 17/3201; A61B 90/00

(52) U.S. Cl. CPC A61B 17/1222; A61B 17/00234; A61B 17/320016; A61B 17/3201; A61B 90/03; A61B 2017/00238; A61B 2017/00353; A61B 2017/1225; A61B 2090/034

(57) ABSTRACT

A system for use in connection with a blood vessel includes a proximal section including a control system and a distal section. The distal section has connected thereto an application device configure to apply one or more ligating clips to the blood vessel to mechanically ligate the blood vessel. The application system is in connection with the control system. The distal section further has connected thereto a cutting device which is configured to mechanically cut tissue, including the blood vessel (for example, after compressive force is applied to the vessel via one or the one or more ligating clips).

### CROSS-REFERENCE TO RELATED APPLICATION

This application claims benefit of U.S. Provisional Patent Application Ser. No. 63/596,463, filed Nov. 6, 2023, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Heart disease is the leading cause of death in the US, with approximately 700,000 people dying yearly from various heart conditions. The most prevalent condition is coronary artery disease (CAD). CAD refers to a disease in which the coronary arteries cannot pump enough blood and oxygen into the heart because they are blocked or thinned by plaque. In 2020, nearly 400,000 Americans were killed by CAD. Coronary artery bypass grafting (CABG) is a surgical method for treating CAD and preventing further heart risks. CABG is performed by harvesting a vein, most commonly the long saphenous vein in the leg, from elsewhere in the body and using it to bypass the affected artery. Endoscopic vein harvesting or EVH is a common procedure performed to remove the greater saphenous vein (GSV) for use in the CABG procedure. Current tools used in EVH are electrocautery-based harvesting systems for cauterizing and ligating (that is, closing off) vessels and branches to release the saphenous vein. While cauterization can effectively seal off small blood vessel branches, it can fail to fully seal larger branches as a result of incomplete seals across the large branch diameter. Further there is a risk of thermal damage to surrounding tissue and the target vessel itself. Any degree of damage to the vein can increase its risk of failure which would result in rehospitalization and possible reoperation. The limitations associated with cauterizing and ligating vessels can, for example, result in blood loss and patient discomfort, and often necessitate further invasive care.

### SUMMARY

A system for use in connection with a blood vessel includes a proximal section including a control system and a distal section. The distal section has connected thereto an application device configure to apply one or more ligating clips to the blood vessel to mechanically ligate the blood vessel. The application system is in connection with the control system. The distal section further has connected thereto a cutting device which is configured to mechanically cut tissue, including the blood vessel (for example, desirably after compressive force is applied to the vessel via one or the one or more ligating clips). The proximal section and the distal section may, for example, be portions of an extending tube configured for use in endoscopic surgery. In a number of embodiments, the cutting device includes surgical scissors including a first blade which is pivotable relative to a second blade.

In a number of embodiments, the application device includes a first compressive arm and a second compressive arm. The ligating clip is positionable between the first compressive arm and the second compressive arm to compress the ligating clip to ligate the blood vessel.

In a number of embodiments, the application device includes a housing including a compartment which is configured to hold a plurality of the ligating clips. The application device is further configured to apply two or more of the plurality of ligating clips sequentially in time. The application device may, for example, include a clip biasing system to bias the plurality of ligating clip to move in a defined direction.

Each of the plurality of ligating clips may, for example, include a distal resilient section and a proximal clamping section. The clamping section include a first tissue contacting member and a second tissue contacting member. The distal resilient section is attached to the proximal clamping section such that compression of the distal resilient section causes separation of the first tissue contacting member and the second tissue contacting member.

The compartment of the housing of the application device may be in connection with a passage through which one of the plurality of ligating clips may be forced. Forcing the one of the plurality of clips through the passage causes compression of the distal resilient section.

The system may further include a biasing system in connection with the surgical scissors which is configured to bias at least one of the first blade and the second blade to an open state. In a number of embodiments, the cutting device further includes a scissors compartment into which and out of which at least a portion of the surgical scissors may be moved. Moving the at least a portion of the surgical scissors distally into the scissors compartment causes the first blade and the second blade to be forced toward a closed state via abutment of at least of the first blade and the second blade with the scissors compartment. Moving the surgical scissors proximally out of the compartment causing the surgical scissors to be forced toward the open state via the biasing system in connection with the surgical scissors.

In a number of embodiments, the distal section of the system is movable relative to a proximal section to change the angle of the distal section relative to the proximal section. The distal section may, for example, be pivotable relative to a longitudinal axis of the system via a mechanical joint.

A method of cutting and ligating a blood vessel includes causing a ligating clip to be applied to and mechanically compressed around the blood vessel using an application device of a system. The system include a proximal section and a distal section. The application device is in connection with the distal section. The method further includes, after causing the ligating clip to be applied to and mechanically compressed around the blood vessel, mechanically cutting the blood vessel at a predetermined position using a cutting device of the system in connection with the distal section. The system may further include a control system connected to or in operative connection with the proximal end which is in operative connection with the application device and the cutting device. The proximal section and the distal section may, for example, be portions of an extending tube configured for use in endoscopic surgery. In a number of embodiments, the cutting device includes surgical scissors including a first blade which is pivotable relative to a second blade.

In a number of embodiments, the application device includes a first compressive arm and a second compressive arm. The ligating clip is positionable between the first compressive arm and the second compressive arm to compress the ligating clip to ligate the blood vessel.

In a number of embodiments, the application device includes a housing including a compartment which is configured to hold a plurality of the ligating clips. The application device is further configured to apply two or more of the plurality of ligating clips sequentially in time. The application device may, for example, include a clip biasing system to bias the plurality of ligating clip to move in a defined direction.

Each of the plurality of ligating clips may, for example, include a distal resilient section and a proximal clamping section. The clamping section include a first tissue contacting member and a second tissue contacting member. The distal resilient section is attached to the proximal clamping section such that compression of the distal resilient section causes separation of the first tissue contacting member and the second tissue contacting member.

The compartment of the housing of the application device may be in connection with a passage through which one of the plurality of ligating clips may be forced. Forcing the one of the plurality of clips through the passage causes compression of the distal resilient section.

The system may further include a biasing system in connection with the surgical scissors which is configured to bias at least one of the first blade and the second blade to an open state. In a number of embodiments, the cutting device further includes a scissors compartment into which and out of which at least a portion of the surgical scissors may be moved. Moving the at least a portion of the surgical scissors distally into the scissors compartment causes the first blade and the second blade to be forced toward a closed state via abutment of at least of the first blade and the second blade with the scissors compartment. Moving the surgical scissors proximally out of the compartment causing the surgical scissors to be forced toward the open state via the biasing system in connection with the surgical scissors.

In a number of embodiments, the distal section of the system is movable relative to a proximal section to change the angle of the distal section relative to the proximal section. The distal section may, for example, be pivotable relative to a longitudinal axis of the system via a mechanical joint.

A clip for use in connection with a blood vessel includes an opening via which the clip can be placed around the blood vessel, one or more cutting edges positioned to be brought into contact with and cut through tissue of the blood vessel upon compression of the clip around the blood vessel; and one or more ligating sections positioned to be brought into contact with and ligate tissue of the blood vessel upon compression of the clip around the blood vessel, whereby the clip is adapted to both cut through and ligate the blood vessel upon compression of the clip around the blood vessel.

The clip (or a portion thereof) may, for example, be U-shaped. In a number of embodiments, the clip includes a first cutting edge on one leg of the U-shape thereof and a second cutting edge on another leg of the U-shape thereof. The first cutting edge and the second cutting edge are configured to slide past each other during compression of the clip. The clip may further include a first ligating section on one leg of the U-shape thereof and another ligating section on another leg of the U-shape thereof.

In a number of embodiments, the clip is formed from one or more biodegradable materials. Compression of the clip may, for example, occurs solely through application of mechanical force. In a number of embodiments, the clip is configured to cooperate with an applicator tool to be compressed via mechanical force applied manually by a user.

A method of cutting and ligating a blood vessel includes compressing a clip around the blood vessel, the clip including an opening via which the clip can be placed around the blood vessel, one or more cutting edges positioned to be brought into contact with and cut through tissue of the blood vessel upon compression of the clip around the blood vessel, and one or more ligating sections positioned to be brought into contact with and ligate tissue of the blood vessel upon compression of the clip around the blood vessel, whereby the clip is adapted to both cut through and ligate the blood vessel upon compression of the clip around the blood vessel. The clip may, for example, be formed from one or more biodegradable materials.

In a number of embodiments, the clip (or a portion thereof) is U-shaped. The clip may, for example, include a first cutting edge on one leg of the U-shape thereof and a second cutting edge on another leg of the U-shape thereof. The first cutting edge and the second cutting edge are configured to slide past each other during compression of the clip. The clip may further include a first ligating section on one leg of the U-shape thereof and another ligating section on another leg of the U-shape thereof.

In a number of embodiments, the clip is compressed using an applicator system. A distal section of the applicator system may, for example, be movable relative to a proximal section of the applicator tool to change the angle of the distal section of the applicator tool relative to the proximal section of the applicator tool.

In a number or of embodiments, compression of the clip occurs solely through application of mechanical force. The applicator system may, for example, be configured to mechanically compress the clip via mechanical force applied manually by a user.

A system for use in applying a ligating clip to a blood vessel includes a distal section including an application device configured to apply the ligating clip. The distal section is movable relative to a proximal section of the system to change the angle of the distal section of the system relative to the proximal section of the system.

A system for use in connection with a blood vessel includes a proximal section including a control system and a distal section. An application device is in connection or operative connection with the distal section to apply one or more ligating clips to the blood vessel to mechanically ligate the blood vessel. The application device is in connection with the control system. The application device includes a housing including a compartment which is configured to hold a plurality of the ligating clips. The application device is configured to apply two or more of the plurality of ligating clips sequentially in time.

A system for use in connection with a blood vessel includes a proximal section including a control system, a distal section, and a cutting device in connection or operative connection with the distal section. The cutting device is configured to mechanically cut tissue (including the blood vessel). The cutting device include surgical scissors including a first blade which is pivotable relative to a second blade. The cutting device further includes a scissors compartment into which and out of which at least a portion of the surgical scissors may be moved. Moving the at least a portion of the surgical scissors distally into the scissors compartment causes the first blade and the second blade to be forced toward a closed state via abutment of at least one of the first blade and the second blade with the scissors compartment. Moving the surgical scissors proximally out of the compartment causes the surgical scissors to be forced toward the open state via a biasing system in connection with the surgical scissors.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a top plan view of an embodiment of a cutting and ligating clip hereof in an open state.

FIG. 1B illustrated an isometric view of the clip of FIG. 1A in an open state.

FIG. 1C illustrated a rear view of the clip of FIG. 1A in an open state.

FIG. 1D illustrated a side view of the clip of FIG. 1A in an open state.

FIG. 1E illustrated side view of the clip of FIG. 1A in an open state and in operative connection with an applicator tool, applicator device or applicator system.

FIG. 1F illustrated side view of the clip of FIG. 1A in a closed state.

FIG. 1G illustrated schematically a side view of the clip of FIG. 1A in a closed state after use in cutting and ligating a blood vessel.

FIG. 2A illustrates schematically a side view of an embodiment of a system which includes a distal end section having a clip application device or mechanism including pivoting members which are configured to seat a clip therein and apply a compressive force thereto during ligation, and a cutting device or mechanism for cutting a blood vessel and/or tissue.

FIG. 2B illustrates schematically a side view of the system of FIG. 2A in which a distal section of system is moved (for example, rotated or angled) relative to a proximal section thereof.

FIG. 3A illustrated an isometric view of another embodiment of a system hereof which includes an applicator device or mechanism configured to apply a clip to a vessel and a cutting device or mechanism to cut a blood vessel and/or tissue.

FIG. 3B illustrates another isometric view of the system of FIG. 3A illustrating aspects of the operation thereof.

FIG. 4A illustrates an isometric view of the clip application device of the system of FIG. 3A disconnected from the system.

FIG. 4B illustrates schematically a top view of the clip application device with a model of a clip being passed through a forward opening thereof to compress a rearward section of the clip and open a forward, clamping section of the clip.

FIG. 4C illustrates a top view and a forward end view (right side) of a tissue contacting section of the forward clamping section of the clip, wherein La=10 mm, Lb=5 mm, Lc=2 mm, and Ld=0.635 mm.

FIG. 4D illustrates an enlarged isometric view of an embodiment of a clip for use in connection with the clip application device of the applicator system of FIG. 3A.

FIG. 4E illustrates the resilient and malleable material of the clip (before it is formed into the shape or conformation of the clip of FIG. 4D) which was laser cut to a flat conformation.

FIG. 5A illustrates a photograph of a clip of FIG. 4D compressed around a blood vessel having a diameter of approximately 10 mm.

FIG. 5B illustrates a photograph of a clip of FIG. 4D compressed around a blood vessel having a diameter of approximately 2 mm.

FIG. 6 illustrates equations used in modelling forces required to push a clip through the clip application device of the applicator system of FIG. 3A based upon the clip model of FIG. 4B.

FIG. 7A illustrates an isometric view of the embodiment of the cutting device of the system of FIG. 3A disconnected from the system.

FIG. 7B illustrates top view photograph of an embodiment of the cutting device used in a number of studies hereof.

FIG. 7C illustrates a top-view, free-body diagram of a cutting device hereof.

FIG. 8 illustrates equations used in modelling a cutting device hereof based upon the free-body diagram of FIG. 7C.

### DESCRIPTION

The present devices, systems, methods and compositions, along with the attributes and attendant advantages thereof, will best be appreciated and understood in view of the following description taken in conjunction with any accompanying drawings.

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an edge” includes a plurality of such edges and equivalents thereof known to those skilled in the art, and so forth, and reference to “the edge” is a reference to one or more such edges and equivalents thereof known to those skilled in the art, and so forth. Recitation of ranges of values herein are merely intended to serve as a shorthand method of referring individually to each separate value falling within the range. Unless otherwise indicated herein, each separate value as well as intermediate ranges are incorporated into the specification as if it were individually recited herein. Use of the term “approximately”, “about” and the like in connection with a value means within 10% (and more typically within 5%) of the value unless the context clearly dictates otherwise. All methods described herein can be performed in any suitable order unless otherwise indicated herein or otherwise clearly contraindicated by the text.

In a number of embodiments, devices, systems and method hereof enable EVH of, for example, the greater saphenous vein or GSV to provide for a healthier vein extraction and a more time efficient approach to CABG than provided under current methodologies. In a number of embodiments, devices, systems, and methods hereof provide cutting and ligation of a blood vessel (that is, a vein or artery). Devices, systems, and method hereof may, for example, use a mechanical approach for cutting and ligating blood vessels (for example, in EVH). The devices, systems, and methods hereof may, for example, reduce or eliminate the technical and clinical limitations associated with endoscopic electrocautery. Although a number of representative examples of devices, systems, and methods hereof are discussed in connection with endoscopic surgery such as EVH, one skilled in the art will appreciate that the devices, systems, and methods hereof may be used in connection with any surgical procedure involving cutting and ligating of any blood vessel. As used herein, endoscopic surgery refers to surgery using an endoscope, which includes an extending, flexible tube with a camera and light that allows a physician to see inside the body without the requirement of large incisions. During a currently practiced endoscopic vein harvesting surgical procedure, for example, small incisions are made and an endoscopic camera is used like a blunt dissector to create a subcutaneous tissue tunnel under the skin.

In a number of embodiments hereof, surgical clips hereof are suitable to withstand blood flow pressure of at least 50 mmHg. Desirably, clips hereof and applicator devices, systems, or tools used therewith provide for a surgical procedure of 20 minutes or less. In a number of embodiments, applicator tools hereof may be operated, in large part, with a single hand. In a number of embodiment, applicator tools hereof may desirably operate within approximately a 20 mm diameter passage associated with dimensional constraints in harvesting via EVH the GSV. Materials of clips and applicator tools hereof should be biocompatible. Desirably, applicator tools hereof are suitable to sever perpendicular and parallel veins and tissue up to at least 10 mm in diameter/thickness. In a number of embodiments, applicator tools hereof are suitable to sequentially implant a plurality of clips.

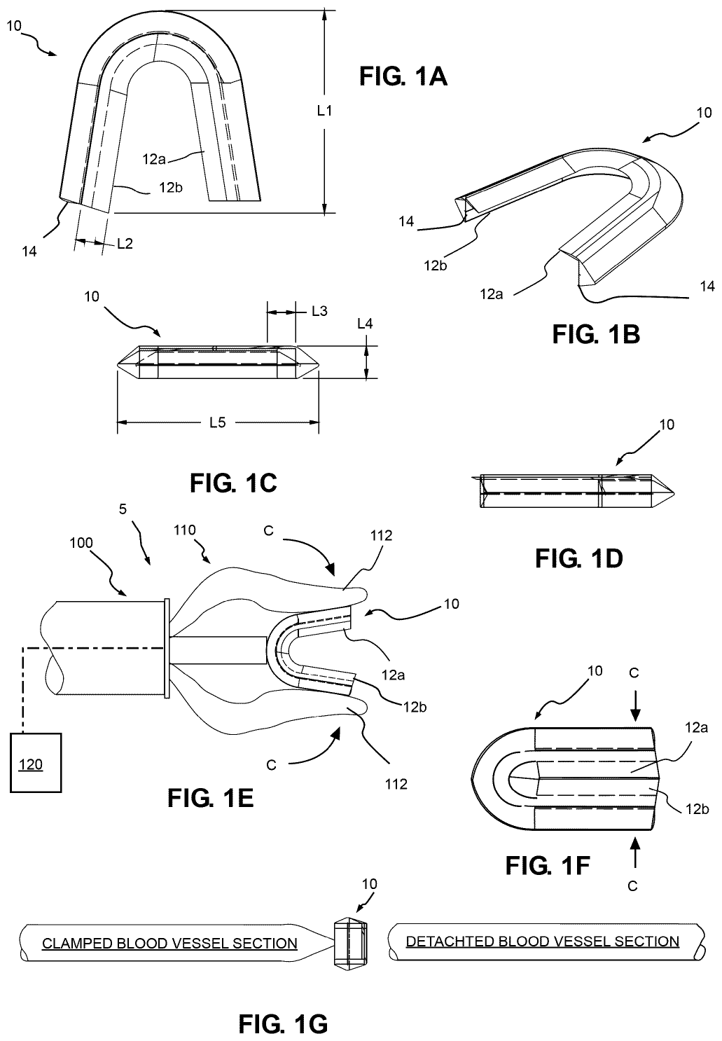

A representative embodiment of a device, surgical clip, or clip 10 suitable to mechanically achieve both cutting and ligation of a vessel is illustrated in FIGS. 1A through 1G. Representative examples of dimensions are provided in FIGS. 1A and 1C (wherein, in mm, L1=5.66, L2=0.79, L3=0.80, L4=0.90, and L5=5.63). Similar to existing ligation clips for vessel ligation, clip 10 may be generally C- or U-shaped and is formed of a flexible or malleable material such that clip 10 can be closed, crimped, or clamped to a shut position around a vessel (see, for example. FIGS. 1F and 1G). Unlike currently available ligation clips, however, clip 10 includes one or more sharpened or pointed ends, edges, sections, or blades for cutting through a blood vessel. In the illustrated embodiment, clip 10 includes a first sharpened cutting blade 12a and a second sharpened cutting blade 12b which are configured or adapted to cut through a blood vessel during closure of clip 10 and sever the blood vessel at the point of contact therewith. As illustrated in the embodiment of FIGS. 1A through 1F, blades 12a and 12b are formed separately on each leg of U-shaped clip 10 and are forced together via compressive force from applicator 100 while clip 10 is positioned such that the vessel is positioned within the opening of U-shaped clip 10. Blades 12a and 12b may, for example, be wedge-shaped, coming to a point at the inner ends thereof where clip 10 contacts the blood vessel. Clip 10 also includes one or more ends, edges, or sections 14 (positioned adjacent cutting edges or blades 12a and 12b in the illustrated embodiment) used to ligate the cut vessel by clamping it shut when clip 10 is fully compressed around the blood vessel. In general, the ligating/clamping ends, edges, or sections 14 are not sharpened and may, for example, be generally flat or curved. Upon closing, the blades 12a and 12b will slide over each other to perform a cutting motion akin to the blades on a pair of scissors, allowing ends, edges, or sections 14 to contact each other and clamp shut upon full closure. Cutting edges or blades 12a and 12b will lie on top of each other or overlap after the compressing motion to ensure that there is no exposure of a sharpened edge or blade that could damage anything nearby.

Clip 10 and other clips hereof may, for example, be formed from a metal such as stainless steel or titanium. Clip 10 may also be formed from a biodegradable material such as a biodegradable polymer, a biodegradable metal, a biodegradable metal alloy, or combinations thereof. Biodegradable metallic materials suitable for use in clips 10 include, for example, zinc, copper, magnesium, iron and metal alloys including such metals. Biodegradable polymers for use in clips 10 include, for example, poly(d,l-lactide-co-trimethylene carbonate), lactide-glycolide copolymer and polyglycolide, poly lactic acid/calcium peroxide composite, poly-(lactic acid), and polymerized high internal phase emulsions (PolyHIPEs).

FIG. 1E illustrates schematically a side view of a portion of a representative embodiment of a tool, instrument or system 100 for use in connection with clip 10. System 100 operates in a similar manner to applicator systems used in connection with existing ligation clips. In that regard, system 100 includes a distal end section including a clip application device 110 including pivoting members 112 which are configured to seat clip 10 therebetween and apply a compressive force thereto (represented by arrows C in FIGS. 1E and 1F) to compress clip 10 around the blood vessel (upon actuation of an actuator or control system 120 by the user (for example, a control system of an endoscopic system); see FIG. 1E). A blood vessel that has been simultaneously or contemporaneously (that is, occurring within a short period of time) cut and ligated using a system 5 (see FIG. 1E) including tool, instrument or system 100 and clip 10 is illustrated in FIG. 1G in which the rearward end of clip 10 is visible. As discussed further below, a plurality of clips 10 may, for example, be stored within a housing (not shown) of system 100 for serial application to blood vessels. Two or more of such stored clips may be delivered sequentially in time to pivoting end members 112 for application.

Clip 10 significantly expands upon existing surgical clip designs by incorporating one or more sharpened or cutting edges, ends, sections or blades (for example, cutting blades 12a and 12b in the illustrated embodiment) designed to cut tissues in addition to a duller or flatter ends or edges that function as a clamp for ligating the cut vessel. The small size and simple design of the clip 10 hereof results in relatively low production costs. Clip 10 also provides for increased precision and control as compared to current techniques, thereby improving safety outcomes of EVH (for example, by reducing operation times and the degree of invasiveness) and other procedures in which a blood vessel (vein or artery) must be cut and ligated.

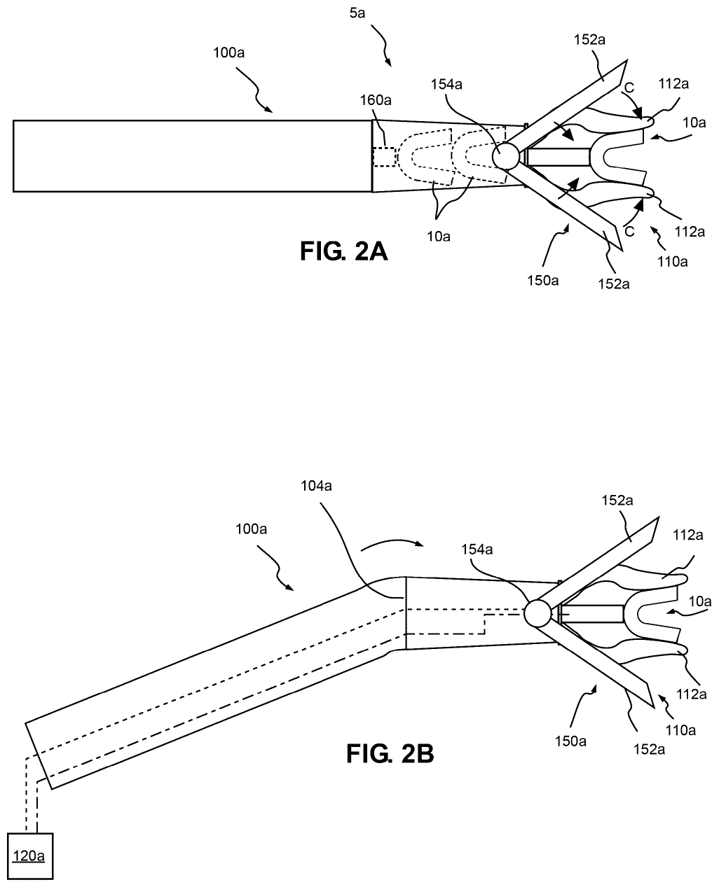

FIGS. 2A and 2B illustrates a representative embodiment of a system 5a hereof including a tool, instrument or system 100a and a clip 10a in operative connection with system 100a. Clip 10a may, for example, be a currently available ligation clip as known in the art. Unlike clip 10, clip 10a need not include a cutting edge. In the embodiment of FIGS. 2A and 2B, system 100a includes a distal end section including an application device 110a which includes pivoting members 112a which are configured to seat clip 10a therebetween and apply a compressive force thereto (represented by arrows C in FIG. 2A) to compress clip 10a around the blood vessel upon actuation of an actuator or control system 120a (as described above for applicator 100 and clip 10; see FIG. 2B). As discussed further below, a plurality of clips 10a may, for example, be stored within applicator system 100a (see. FIG. 2A) for serial application to blood vessels. Two or more of such stored clips may be delivered sequentially in time to pivoting end members 112a of the application device 110a to be applied to a blood vessel via a biasing mechanism 160a (see FIG. 2A) as described further below. Upon ligation of the vessel via clamping application of clip 10a, a cutting mechanism or blood vessel cutting device 150a may be actuated by the user via actuator or control system 120a (see FIG. 2B). In the illustrated embodiment, tissue/blood vessel cutting device 150a is formed to operate in the manner of surgical scissors and include blades 152a (positioned on or in the vicinity of the distal section of system 100a) that pivot about a pivot connector 154a.

As illustrated in FIG. 2B, a distal section of system 100a may be moved (for example, rotated, angled or pivoted) via, for example, a pivoting mechanical joint 104a (represented schematically in FIG. 2B) relative to a proximal section (for example, via actuator or control system 120a) to facilitate position of the distal section of system 100a. System 100, as discussed above for use in connection with clips 10, may be similarly controlled to facilitate a position of the distal section thereof relative to the proximal section.

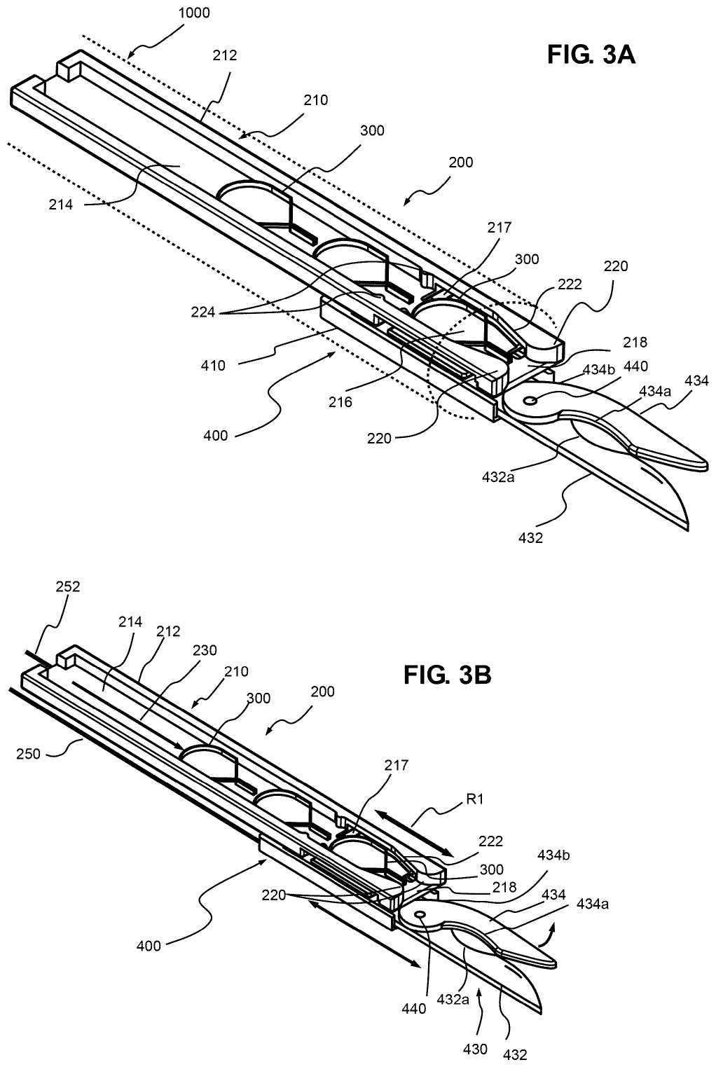

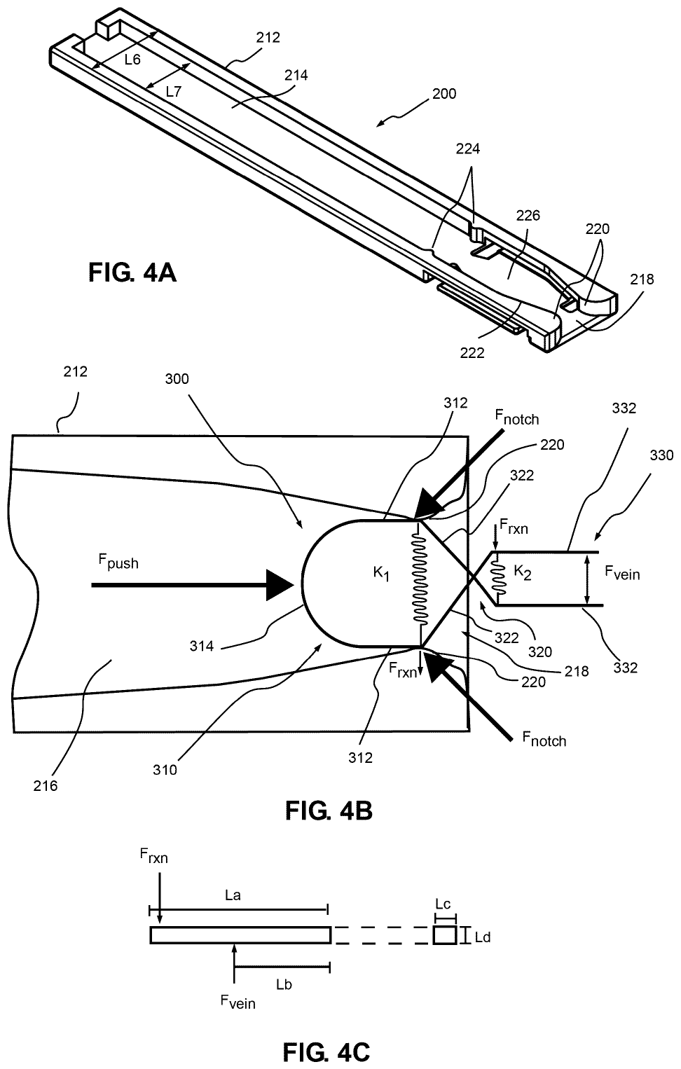

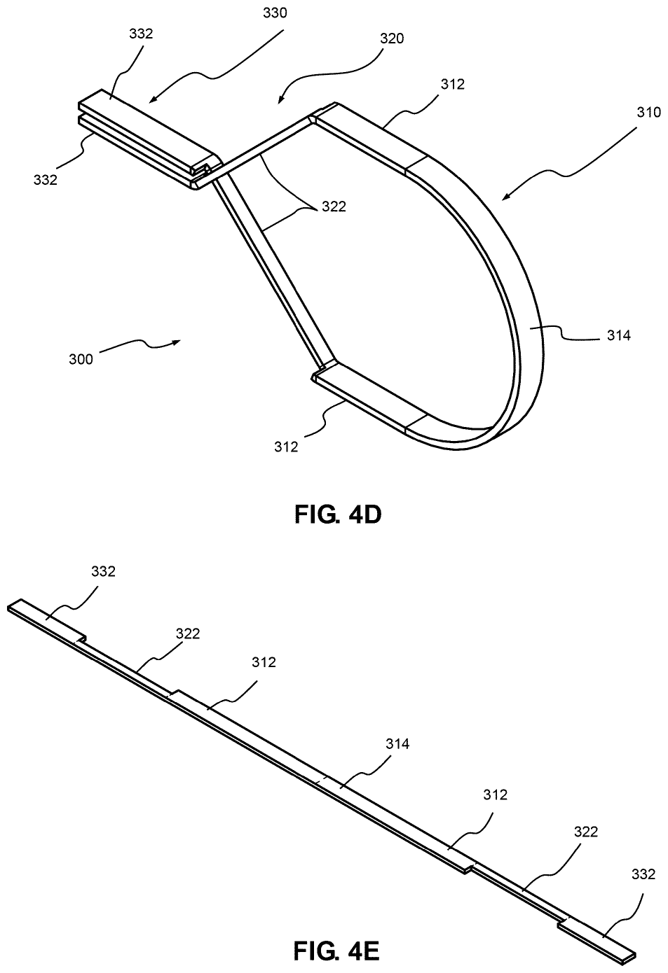

FIGS. 3A and 3B illustrate another embodiment of a tool, instrument or system 200 hereof which (like system 100 and 100a) may be a component of an endoscopic system probe 1000 illustrated schematically in broken lines in FIG. 3A. System 200 includes a clip application mechanism or device 210 (see, for example, FIGS. 3A through 4A) via which one or more clips 300 (as, for example, illustrated in FIGS. 3A and 4B through 4D) can be applied to tissue. System or applicator 200 further includes a tissue cutting mechanism or device 400.

In the illustrated embodiment, application device 210 includes a housing or body 212 that includes a volume, chamber or compartment 214 into which multiple clips 300 may be loaded. In the illustrated embodiment, compartment 214 is formed as a longitudinally extending channel, but other conformations are possible. Compartment 214 operates similar to a magazine used to load and deliver cartridges to a chamber. In that regard, compartment 214 may, for example, house multiple clips 300 and provide for application of such clips 300 in series or succession onto veins ranging from, for example, 2 mm to 20 mm, or 2 mm to 10 mm in diameter.

In the illustrated embodiment of FIGS. 4B through 4E, clip 300 may, for example, include a rearward or distal section 310, an intermediate section 320, and a forward, proximal or blood vessel contacting/clamping section 330. In a number of studies, clip 300 was formed from an integral or monolithic length of metal as illustrated in FIG. 4E by controlled laser cutting of a flat length of the metal from a metal sheet and bending the length of metal into formed clip 300 as illustrated, for example, in FIG. 4D. Reward section 310, which is formed as a resilient, compressible loop, includes two generally linear or flat, longitudinally extending sections 312 which are interconnected at a rearward end thereof to a curved section 314 in the illustrated embodiment. The forward ends of extending sections 312 are connected to rearward ends of extending cross members 322 of intermediate section 320. As illustrated in FIGS. 4D and 4E, extending cross members 322 were approximately one half the width of the remainder of clip 300 such that extending members 322 could cross while maintaining a generally planar and shallow/thin conformation of clip 300.

The forward ends of extending cross members 322 are connected to rearward ends of longitudinally extending tissue contacting members 332 of forward (or proximal) blood vessel contacting/clamping section 330. The forward end of tissue contacting members 332 are free ends such that compression of extending sections 312 toward each other results in movement of tissue contacting members 332 away from each other and “opening” of blood vessel contacting/clamping section 330 to receive a blood vessel between tissue contacting members 332. Clamping forces is exerted upon the blood vessel, which compression force is removed from rearward section 310 (for example, from one or both of extending sections 312) such that rearward section expands and tissue contacting members 332 are forced toward each other.

Referring, for example, to FIGS. 3A through 4B, during application to a blood vessel a clip 300 is forced from an application compartment, section or chamber 216, which is positioned on a forward end of compartment 214, through a passage or opening 218 (see, for example, FIGS. 3A through 4A) which is defined by inward projecting flanges or shoulders 220 on each side of application chamber 216. Shoulders 220 may, for example, include gradually inward sloping surfaces 222 which contact clip 300 and compress rearward section 310 thereof as clip 300 is pushed forward within application chamber 216. As illustrated in FIG. 4B, contact of surfaces 222 with clip 300 can, for example, occur at the connection of extending sections 312 and extending cross members 322. As described above, compression of extending sections 312 toward each other results in movement of tissue contacting members 332 away from each other and opening of blood vessel clamping section 330. Once clip 300 is forced through opening 218, compression force is removed from rearward section 210, such that tissue contacting members 332 are forced toward each other and clamping force is applied to the blood vessel.

In a number of embodiments, compartment 214 of clip applicator device 200, operates in the manner of a “plunger” to advance one of surgical clips 300 positioned therein forward into application chamber. In that regard, clips 300 may be pushed by a biasing element such as a spring, elastomeric element, or other resilient element 230 (represented schematically as an arrow in FIG. 3B) for the primary linear force in contact with the clips 300. As a force is applied from the rear via biasing element 230, a longitudinally extending first rigid member 250, such as a rod or wire of suitable stiffness (represented schematically in FIG. 3B), may be used to supply additional forward oriented force to push a clip 300 along the tapered edges 222 of application compartment 216 and through opening 218 as described above, thereby clamping onto the targeted blood vessel. First rigid member 250 thereby controls the application of clips 300. In a number of embodiments, when a user applies forward force to first rigid member 250, first rigid member 250 enables a clip 300 to be applied from application chamber 216 into position for use on a vessel as described above. After clip 300 is applied from application chamber 216, biasing element 230 applied forward force to clip(s) 300 in compartment 214 and “reloads” application chamber 216 with next clip 300, allowing for seamless reapplication without manual intervention. In a number of embodiments, first rigid member 250 operates as both an application mechanism and a blocker to control the flow of clips 300 in application device 210 of system 200. Initially, first rigid member 250 is in a retracted position, in which it may block biasing element 230 from pushing subsequent clips 300 into application chamber 216. In a number of embodiments, first rigid member 250 may include or be in operative connection with an abutment member (not shown) via a passage or opening 217 in a lower surface of application chamber 216. When the user applies forward force to first rigid member 250, it disengages the abutment member from its blocking position (sliding it forward, out of its blocking position and out of opening 216), allowing biasing element 230 to apply forward force to clip 300 loaded with application chamber 216 to push it out of application chamber 216 for application to a vessel. As first rigid member 250 continues forward, clip 300 is applied to the target area/vessel. Simultaneously, biasing element 230 advances next clip 300 into application chamber 216. Upon applying pulling or rearward force to first rigid member 250, first rigid member 250 returns to its original, blocking position, once again blocking next clip 300 from forward motion and thereby preventing multiple clips 300 from being pushed out by biasing member 230. The described functionality ensures that only one clip 300 is loaded into application chamber 216 and applied at a time, while biasing member 230 keeps remaining clips 300 ready for the next cycle. Control of movement of first rigid member 250 thus coordinates timing and placement of each clip 300. Pulling back on a handle or grip in operative connection with first rigid member 250 resets application chamber 216 for the next cycle. The motion of first rigid member 250 is represented schematically by arrow RI in FIG. 3B.

In a number of embodiments, clips 300 were constructed from a biocompatible and malleable material such as a metal (for example, stainless steels such 301 Full Hard Tempered Stainless Steel, certified pure titanium, an alloy, etc., as known in the medical instrument arts). Like clip 10, clip 300 may also be formed from a biodegradable material such as a biodegradable polymer, a biodegradable metal, a biodegradable metal alloy, or combinations thereof. To begin evaluating such metals, they were first laser cut into the desired flat pattern for surgical clips 300 as illustrated in FIG. 4E. Next, the flat-patterned materials were manually bent into the desired geometry as illustrated, for example, in FIG. 4D. Upon testing clips 300 formed from stainless steel or titanium, it was apparent that both materials could elastically deform and apply a significant amount of pressure upon closure. In a number of studies, the clips were placed on real veins that were pressurized. Clips formed from either full hard tempered stainless steel or certified pure titanium exceeded minimum established requirements (as describe below) by a significant amount.

Stainless steel was selected for further evaluation in studied hereof. Stainless steel exhibited a slightly higher strength and elasticity than the pure titanium. However, the characteristics of titanium can surpass stainless steel when appropriately alloyed. In that regard, a number of studies have demonstrated that pure and low-alloyed titanium exhibit a lower yield strength and higher average strain than the studied stainless steel. As the alloying content increases, the yield strength of titanium can far surpass that of stainless steel, with the strain deformation characteristic becoming more similar to stainless.

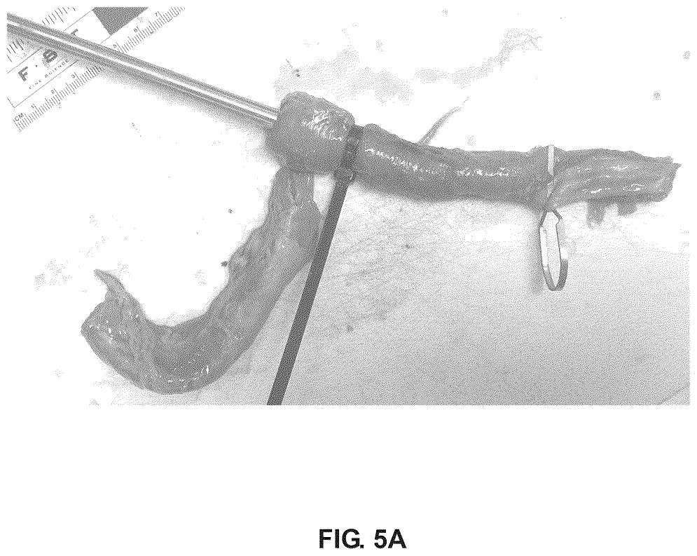

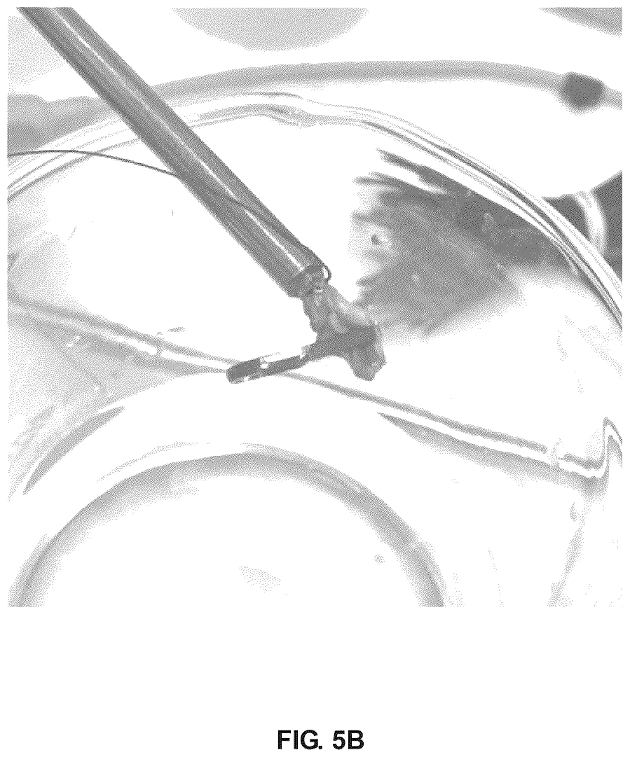

Clips 300 were tested (external from device 210) on varying sizes of porcine aorta veins ranging from approximately 2 mm to approximately 20 mm in diameter. Excess tissue was cut away from the veins. Once the various sized veins were isolated, clips 300 were applied to the varying sample sizes, and the veins were injected with fluid to build up pressure, which was measured via a state-of-the-art liquid pressurization device that tracked changes in real-time. FIG. 5A illustrates a clip 300 in clamping connection with a vein having a diameter of approximately 10 mm, while FIG. 5B illustrates a clip 300 in clamping connection with a vein having a diameter of approximately 2 mm. As described above, a goal of clips hereof is to exceed a minimum fluid pressure of 50 mmHg, which is the maximum blood flow pressure achieved by a number of veins of interest. Maximums blood flow pressure exceeding 90 mmHg was achieved with a multitude of clip materials and sizes.

Results with clips 300 varied depending on material and plate thickness of the metal used in fabricating clips 300. There was a much higher clamping force with larger metal plate thicknesses. Optimization of dimensions and shapes/conformations of clips 300 can be achieved through analytical modeling as further described below.

In a number of embodiments, an outer dimension or outer width L6 (see, FIG. 4A) of housing or body 212 was approximately 15 mm or less, and an internal dimension or width L7 of extending compartment 214 was approximately 10 mm or less. Thus, device 210 meets the dimensional requirements of a 20 mm diameter and clips 300 meet the requirements of a maximum clamping diameter of 10 mm of the GSV, while maintaining a clamping force powerful enough to resist a minimum pressure of 50 mmHg (as set forth above).

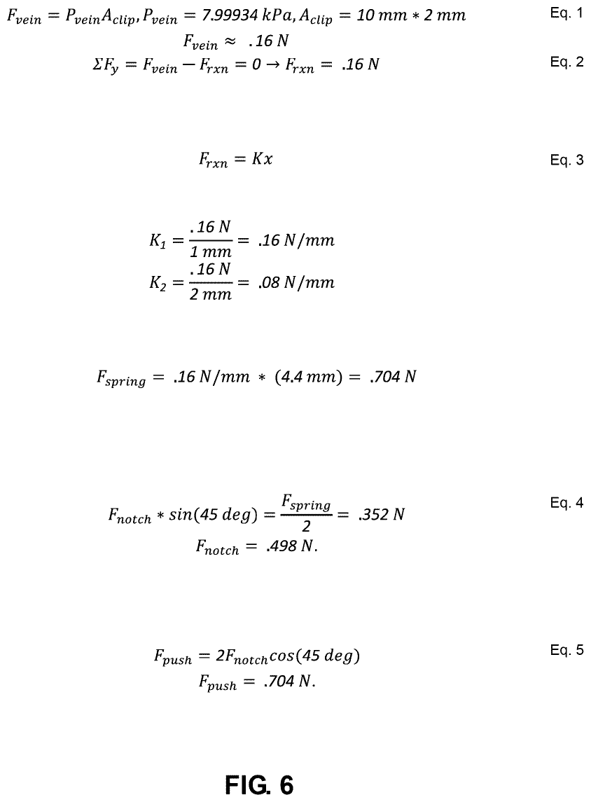

In designing application devices 210 hereof, important considerations include the force required to open a clip 300 and the force required to deliver a clip 300 as it is advanced through application chamber 216 and opening 218. With reference to FIGS. 4B and 4C, FIG. 6 sets forth equations of an analytical model to determine the necessary amount of force required to open a clip 300 and ensure a determined vertical displacement can be met, as well as to determined force required to deliver a clip 300. One may, for example, set forth initial constraints of sustaining at least 60 mmHG (7.99934 kPa) flow pressure from the vein and a maximum of 10 mm in diameter. Given such conservative vein pressure, a force (Fᵥₑᵢₙ) can be calculated acting on (generally flat) tissue contacting sections 332 of clip 300. It may be assumed that the soft tissues of the vein evenly apply the pressure along the whole length of tissue contacting sections 332 of clip 300. As the area of concern is mainly tissue contacting sections 332, they can be isolated and analyzed as beams. Additionally, the symmetry about the midline of clip 300 allows for the analysis to be cut in half as illustrated in FIG. 4C.

To calculate the force from the vein's pressure and the resulting reaction force from the clip, the calculations set forth in Eqs. 1 and 2 used (wherein Pᵥₑᵢₙ is the pressure in the vein, and A_{clip} is the area of tissue contacting section 332). While the force is not located at the pinch point to open clip 300, an assumption can be made that clip 300 acts as two springs in parallel (having spring constants K₁ and K₂) as illustrated in FIG. 4B. When compressing one spring, the other spring is in tension and vice versa. Such behavior is necessary to model in understanding how spring 2 displaces to 10 mm in length. To determine the spring constants, an approximate displacement of 2 mm was measured when testing clips 300 on a vein for spring 2. During the measurement, the length of spring 1 was roughly 9 mm, which corresponds to a 1 mm decrease from its resting length of 10 mm. According to Hooke's Law Fᵣₓₙ=Kx (Eq. 3). Thus K₁ was determined to be 0.16 N/mm, and K₂ was determined to be 0.08 N/mm. Thus, for every millimeter spring 1 displaces, spring 2 will displace two millimeters.

After determining the spring constants as described above, a force (Fₚᵤₛₕ) required to push clip 300 out device 210 can be estimated. FIG. 4B provides a model of delivering a clip 300 from device 200. To determine a required pushing force Fₚᵤₛₕ, the displacement of spring 1 when under compression from each shoulder 220 (F_{notch}) must be taken into account. In several studied embodiments, distance between both shoulders 220 (or the width of opening 218) was approximately 5.6 mm. Therefore spring 1 is experiencing approximately 0.704 N in the vertical direction (in the orientation of FIG. 4B) as determined via application of Eq. 3. This result means that the force from each shoulder 220 is half of what the modeled spring experiences. Using some basic trigonometry, the force from shoulder 220 in the vertical direction can be determined via Eq. 4 as 0.498 N. Thus, the force required to force a clip 300 out of device 210 is 0.704 as determined via Eq. 5, which is a reasonable amount of force required of a physician/surgeon in operating device 210 of system 200. As clear to those skilled din the art, push force Fₚᵤₛₕ can be adjusted via material clip choice, clip design, application chamber design, etc.

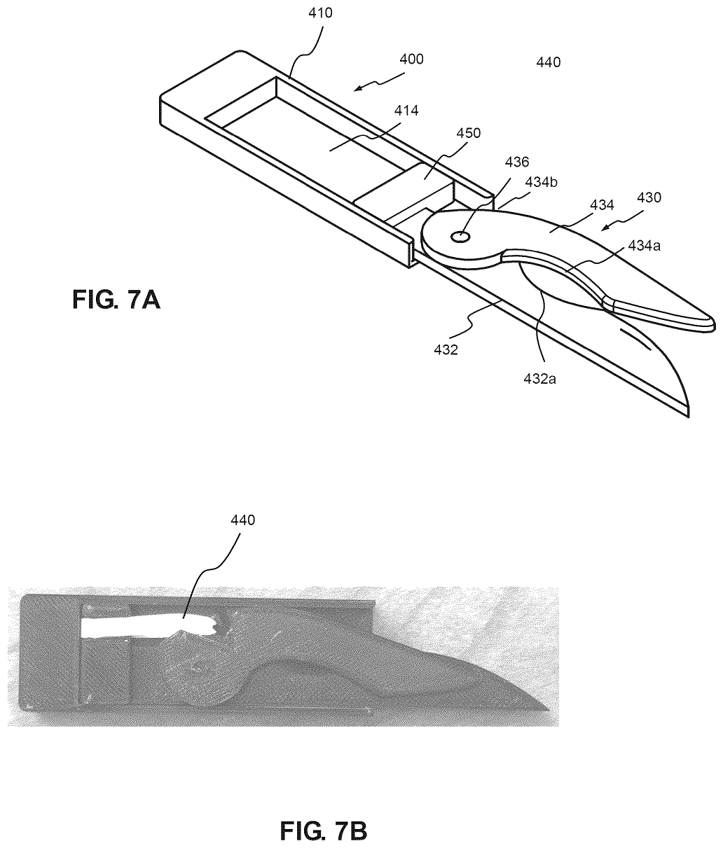

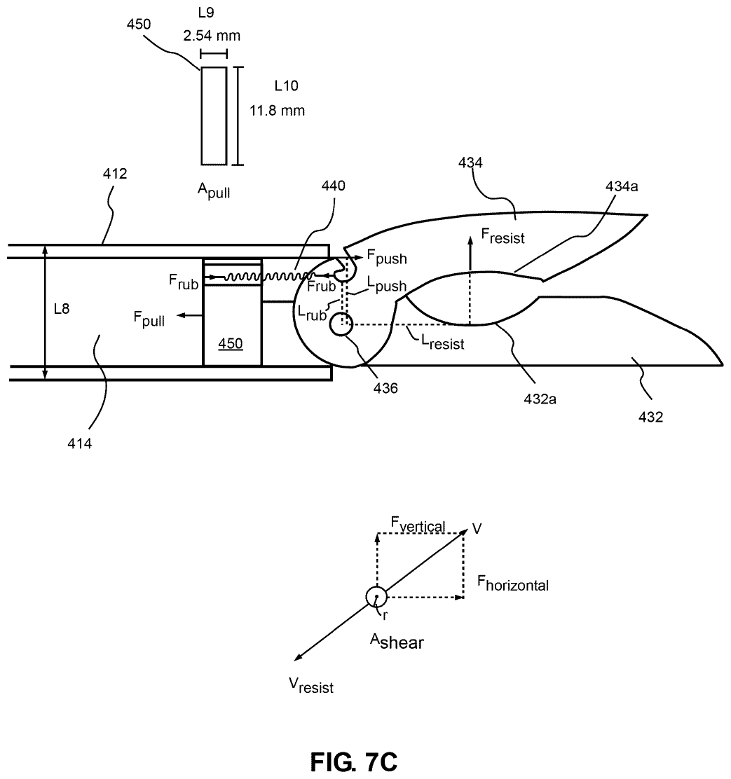

As illustrated in the representative embodiment of FIGS. 3A, 3B. 7A through 7C, cutting device 400 may include a housing or body 410 having a volume, chamber or compartment 414 therein. In the illustrated embodiment, compartment 414 has the form of a longitudinally extending channel. In a number of embodiments, a dimension L8 (for example, designated width: see FIG. 7C) of body 412 was approximately 10 mm. In the illustrated embodiment, a tissue/blood vessel cutter 430 is formed to operate in the manner of surgical scissors and includes blades 432 and 434 that pivot about a pivot connector 436. In the illustrated embodiment, each of blades 432 and 434 is formed with an arced or curved indentation 432a and 434a, respectively, to facilitate a clean slice through veins and tissue. A biasing element or mechanism 440 such as a spring or other resilient element (see FIGS. 7B and 7C) is connected to at least one of blades 432 and 434 (upper blade 434 in the illustrated embodiment) to bias cutter 430 such that blades 432 and 434 revert to an open state after each cut. In the embodiment illustrated in the photograph of FIG. 7B an elastomeric band was used as biasing mechanism 440.

Blades 432 and 434 may be closed onto target tissue by cause blades 432 and 434 to be retracted into compartment 414 as illustrated in FIG. 7B. In the illustrated embodiment, blade 432 is connected to a support 450 which is slidably positioned within a channel of compartment 414. Blade 434 is pivotably or rotatably connected to blade 432 via pivot connector 436 as described above. In the illustrated embodiment, upon retraction of blades 432 and 434 into compartment 414, an arced surface 434b or blade 434 contacts an inner wall of the channel formed by compartment 414 and blade 434 is thereby forced (pivoted) into the closed position relative to blade 432. Support 450 may, for example, be connected to a cable (not shown, but represented schematically as force Fₚᵤₗₗ in FIG. 7C) to exert a pulling or retracting force on support 450 and thereby on blades 432 and 434. Such a methodology of closure/cutting allows cutting device 400 to close relatively effortlessly onto and cut through a blood vessel or tissue.

Referring to FIG. 3B, a second rigid member 252 may be used to control the cutting function of cutting device 400 of system 200. Second rigid member 252 controls the movement of support 450 and thereby cutter 430. Such movement may, for example, require more force than required for clip application as described above. When a user applies forward force to second rigid member 252, it moves support 450 and cutting device 430 forward, out of compartment 414, which results in opening of scissor blades 432 and 434 as described above. As pulling or rearward force is applied to second rigid member 252, blades 432 and 434 are caused to retract to within compartment 4140, forcing/moving blades 432 and 434 toward a closed position, causing cutting of a blood vessel positioned between blades 432 and 434 before complete retraction thereof (that is, the position of FIG. 7B). Both first rigid member 250 and second rigid member 252 may move in a coordinated motion, to operate first and second rigid members 250 and 252 simultaneously, providing for coordinated clipping and ligation of system 200. In a number of embodiments, while rigid members 250 and 252 are moved contemporaneously or simultaneously, rigid member 250 will first disengage from its blocking position as described above, allowing the application of a clip 300 to occur, before cutting device 400 sequentially executes the cut. The application of force by the user may be a single continuous movement, while, temporally, the ligation occurs before the cut.

Studied embodiments of cutting device 210 were demonstrated to sever veins and tissues up to a diameter of at least 10 mm. Such functionality is, for example, necessary for the largest section of the GSV. Cutting device 200 allows a user to finely sever veins and tissues relatively effortlessly while achieving dimensional constraints necessary for EVH of the GSV. The elastomeric (rubber) band was operable to restore blades 432 and 434 to their original position when retracted into housing 410. After some simple movement performed by hand, it was determined that housing 410 was dimensioned suitably to sever veins and tissues completely without concern of slippage. It was further confirmed that the elastomeric band, which operates as biasing element 440, is operable to restore blades 432 and 434 to their optimal position during protrusion.

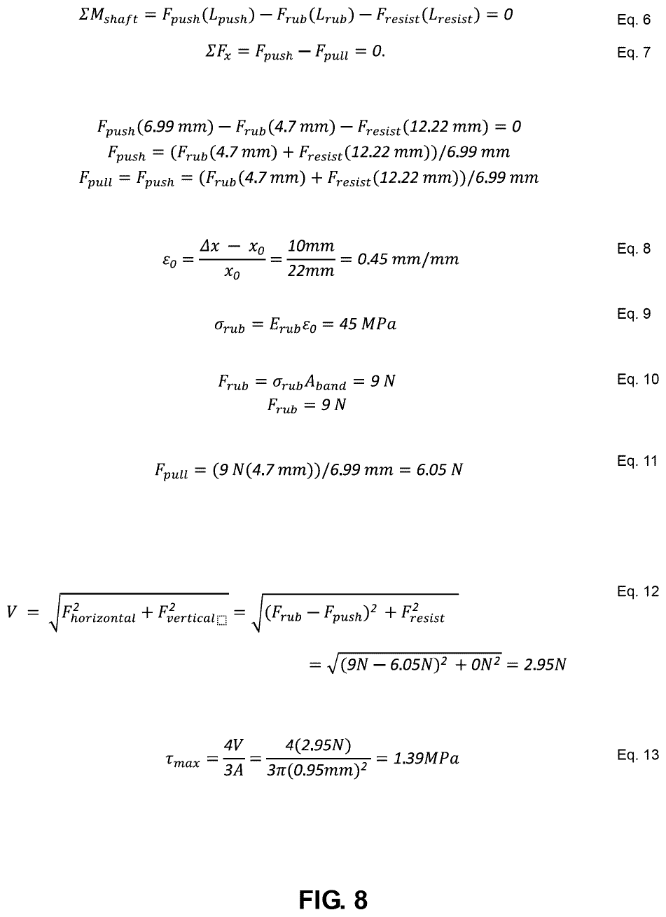

In modeling cutting devices 400 hereof, the force required to retract blades 432 and 434 and to cut the vein/tissue are important parameters. To determine the amount of pull force required to cut, for example, a vein, a diagram of cutting device 400 can be modeled to include the moments caused by biasing mechanism 440, the push force on blade 432 from housing or body 412, and reaction force from the vein. FIG. 7C illustrates a free body diagram of an embodiment of cutting device 400. Cutting device 400 may be analyzed as a static system using the equations of FIG. 8 to estimate a result. Eq. 6 sets forth a momentum balance about the axis/shaft of pivot connector 436, while Eq. 7 sets forth a push/pull force balance. In FIG. 7. L9=2.45 mm, L10=11.8 mm, L_{rub}=4.7 mm, Lₚᵤₛₕ=6.99 mm, Lᵣₑₛᵢₛₜ=12.22 mm, and r=0.95 mm. The measurements from the physical model are inserted into Eqs. 6 and 7 and reduced (as illustrated below Eqs. 6 and 7 in FIG. 7).

After testing, it was quickly determined that veins pose essentially no resistance to scalpel-like blades such as blades 432 and 434. Therefore, the resistance force from the vein will likely be negligible in practice. Further, the force from the rubber band, used as a representative biasing mechanism in studied embodiments, or element 440 must be considered. Given the band's elastic nature, a stress can be determined from its corresponding strain. In its resting position, the length of the band was 22 mm, and when stretched the length was 32 mm. The strain can be calculated as set forth in Eq. 8, which was used to calculate a stress in Eq. 9, assuming a Young's modulus of 0.01 GPa. Given the calculated stress, the cross-sectional area of the rubber band can be utilized to find the corresponding force F_{rub} as set forth in Eq. 10. The force required to retract blades 432, 434 and cut the vessel is calculated in Eq. 11 as 6.05 N. Such a force is readily applied by any medical professional using system 100 and cutting device 400 thereof to perform surgery.

Additionally, the maximum shear pressure was determined at the pivot point of blades 432 and 434 according to Eqs. 12 (sheal force V) and 13 (maximum shear pressure τₘₐₓ) to ensure the material does not fail. The maximum shear pressure on the pivot point was determined to be 1.39 MPa. If, for example, blades 432 and 434 were fabricated using 301 stainless steel, the tensile yield strength of the material is 205 MPa. Assuming that the shear strength is 60% of its tensile yield strength, the shear strength would be approximately 123 MPa. As describe above, the design has a maximum shear pressure of 1.39 MPa, resulting in a safety factor of 88.

Devices and systems hereof provide a cutting mechanism, a clamping mechanism, and a “magazine” to deliver a plurality of clamping clips sequentially. The systems hereof meet dimensional requirements of vein grafting surgery, can sever tissue and veins with a maximum diameter of at least 10 mm, and long-term implantation of a clamping device (in the case of a non-biodegradable clip 300) on a vessel having a diameter of up to, for example, 10 mm, while exceeding the minimum clamping force needed to restrict 50 mmHg of pressurized flow. The devices, systems, and methods hereof employ purely mechanical techniques to harvest and seal the GSV, including its branches, without the need for cauterization.

A system such as system 200 hereof may, as described above, be used as a component of an endoscopic system 1000 as illustrated schematically in FIG. 3A. Such system are readily rotatable about the longitudinal axis thereof. System 200 is also desirably pivotable at an angle to the longitudinal axis as, for example, described in connection with FIG. 2B. Such pivoting motion may, for example, be achieved using a mechanical joint which may provide for articulation of system 200. Mechanisms for controlling such motion of a distal element may readily be adapted from mechanisms used in commercial borescopes. The pivotal aspect is beneficial in achieving efficient access to apply the cut-ligate functionality of a system hereof with minimal maneuvering to access 1) a main vessel, which may extend perpendicular to the system axis/entry direction, and to access 2) non-perpendicular tributaries/branches to the main vessel. Tools, instruments, or systems 100, 100a, and 200 (as well as other systems hereof) may, for example, have the ability to rotate from 0 degrees (for example, oriented parallel to or along the length of the vein) up to 90 degrees or more (oriented perpendicular to the vein). To provide rotational capability, devices and system hereof may, for example, be added to the head or distal end of a borescope or borescope-like device and the two devices can work in unison to create the 180 degrees of freedom desirable for endoscopic vein harvesting. Devices, methods, and systems hereof may, for example, eliminate the need to pull the blood vessel (such as the saphenous vein) out of the body to cut it. In current practice, the blood vessel is pulled out of the body through a second incision so that the vessel can be manually tied off and detached at the end thereof before being returned. Devices, methods, and systems hereof may, for example, provide the means/ability to cut and ligate a main blood vessel (for example, in an EVH procedure) without removing the blood vessel from the body and returning the blood vessel to the body. The performing medical practitioner can, for example, initially angle an applicator system generally parallel to the target vein (for example, the saphenous vein) to begin detaching it. In that regard, applicator system will initially enter parallel to the target vein, which will orient applicator system generally perpendicular to the surrounding tributaries and branches that will need to be cut off. Once the target vein is freed from the branches, the target vein itself must be cut out. The cut at the incision site is relatively simple. However, under current procedures a second incision at the other end of the vein is necessary to fully detach the vein. The movable (for example, pivoting or rotating end) of the applicator system enables the medical practitioner performing the procedure to rotate the distal end or head of the tool to a position to simply cut and clamp the other end of the vein via the initial incision. Subsequently, the vein can be removed from the initial incision without the need of a second incision.

The devices, systems, and methods hereof may, for example, provide a more precise and controlled approach to, for example, detaching the saphenous vein from the surrounding tributaries, reduce the risk of thermal damage to the surrounding tissue, and improve the quality of the harvested vein. The use of mechanical cutting and sealing is practical and may be deployed very similarly to the existing endoscopic electrocautery. The devices, systems, and methods hereof have the potential to significantly improve the safety and outcomes of EVH by reducing operation times, reducing the risk of vessel reopening, and reducing the degree of invasiveness of the procedure.

The devices, systems, and methods, hereof, in a number of embodiments, address the limitations of existing endoscopic electrocautery by providing a 100% mechanical approach as described above. In that regard, in a number of embodiments, the devices, systems, and methods hereof do not require any electricity or other external power sources (beyond manual power). Current harvesting tools for EVH rely on electricity to perform electrocauterization of vein branches. The use of electricity for cauterization carries a risk of damaging nearby tissues if tools are not handled properly. Cutting and/or ligating clips hereof may, for example, be made of titanium or other materials (including biodegradable materials) and pose no risk to nearby tissues. Once again, electrocauterization can, in some cases, fail to fully ligate larger vein branches and blood vessels, resulting in wound reopening and bleeding. Surgical clips and clamp designs in many different procedures have been observed to effectively seal cuts. The devices, systems, and methods hereof have the ability to cut and ligate blood vessels quickly simultaneously or contemporaneously (that is, occurring within a relatively short time frame or period), thereby reducing operating times.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A system for use in connection with a blood vessel, comprising:
a proximal section comprising a control system,
a distal section,
an application device in connection with the distal section to apply one or more ligating clips to the blood vessel to mechanically ligate the blood vessel, the application system being in connection with the control system, and
a cutting device in operative connection with the distal section, the cutting device being configured to mechanically cut tissue including the blood vessel.

2. The system of claim 1 wherein the proximal section and the distal section are portions of an extending tube configured for use in endoscopic surgery.

3. The system of claim 1 wherein the cutting device comprises surgical scissors comprising a first blade which is pivotable relative to a second blade.

4. The system of claim 3 wherein the application device comprises a first compressive arm and a second compressive arm, wherein the ligating clip is positionable between the first compressive arm and the second compressive arm to compress the ligating clip to ligate the blood vessel.

5. The system of claim 3 wherein the application device comprises a housing comprising a compartment which is configured to hold a plurality of the ligating clips, the application device being further configured to apply two or more of the plurality of ligating clips sequentially in time.

6. The system of claim 5 wherein the application device comprises a clip biasing system to bias the plurality of ligating clip to move in a defined direction.

7. The system of claim 6 wherein each of the plurality of ligating clips comprises a distal resilient section and a proximal clamping section, the clamping section comprising a first tissue contacting member and a second tissue contacting member, the distal resilient section being attached to the proximal clamping section such that compression of the distal resilient section causes separation of the first tissue contacting member and the second tissue contacting member.

8. The system of claim 7 wherein the compartment is in connection with a passage through which one of the plurality of ligating clips may be forced, wherein forcing the one of the plurality of clips through the passage causes compression of the distal resilient section.

9. The system of claim 3 comprising a biasing system in connection with the surgical scissors configured to bias at least one of the first blade and the second blade to an open state.

10. The system of claim 9 wherein the cutting device further comprises a scissors compartment into which and out of which at least a portion of the surgical scissors may be moved, moving the at least a portion of the surgical scissors distally into the scissors compartment causing the first blade and the second blade to be forced toward a closed state via abutment of at least of the first blade and the second blade with the scissors compartment, and moving the surgical scissors proximally out of the compartment causing the surgical scissors to be forced toward the open state via the biasing system in connection with the surgical scissors.

11. The system of claim 1 wherein the distal section of the system is movable relative to a proximal section to change the angle of the distal section relative to the proximal section.

12. The system of claim 2 wherein the distal section of the system is movable relative to a proximal section to change the angle of the distal section relative to the proximal section.

13. A method of cutting and ligating a blood vessel, comprising:
causing a ligating clip to be applied to and mechanically compressed around the blood vessel using an application device of a system, the system comprising a proximal section and a distal section, the application device being in connection with the distal section, and
after causing the ligating clip to be applied to and mechanically compressed around the blood vessel, mechanically cutting the blood vessel at a predetermined position using a cutting device of the system in connection with the distal section.

14. The method of claim 13 wherein the proximal section and the distal section are portions of an extending tube configured for use in endoscopic surgery.

15. The method of claim 13 wherein the cutting device comprises surgical scissors comprising a first blade which is pivotable relative to a second blade.

16. The method of claim 15 wherein the application device comprises a first compressive arm and a second compressive arm, wherein the ligating clip is positionable between the first compressive arm and the second compressive arm to compress the ligating clip to ligate the blood vessel.

17. The method of claim 15 wherein the application device comprises a housing comprising a compartment which is configured to hold a plurality of the ligating clips, the application device being further configured to apply two or more of the plurality of ligating clips sequentially in time, and wherein the application device comprises a clip biasing system to bias the plurality of ligating clip to move in a defined direction.

18. The method of claim 17 wherein each of the plurality of ligating clips comprise a distal resilient section and a proximal clamping section, the clamping section comprising a first tissue contacting member and a second tissue contacting member, the distal resilient section being attached to the proximal clamping section such that compression of the distal resilient section causes separation of the first tissue contacting member and the second tissue contacting member, and wherein the compartment is in connection with a passage through which one of the plurality of ligating clips may be forced, wherein forcing the one of the plurality of clips through the passage causes compression of the distal resilient section.

19. The method of claim 15 wherein the cutting device comprises a biasing system in connection with the surgical scissors configured to bias at least one of the first blade and the second blade to an open state, and wherein the cutting device further comprises a scissors compartment into which and out of which at least a portion of the surgical scissors may be moved, moving the at least a portion of the surgical scissors distally into the scissors compartment causing the first blade and the second blade to be forced toward a closed state via abutment of at least of the first blade and the second blade with the scissors compartment, and moving the surgical scissors proximally out of the compartment causing the surgical scissors to be forced toward the open state via the biasing system in connection with the surgical scissors.

20. A clip for use in connection with a blood vessel, comprising:
an opening via which the clip can be placed around the blood vessel;
one or more cutting edges positioned to be brought into contact with and cut through tissue of the blood vessel upon compression of the clip around the blood vessel; and
one or more ligating sections positioned to be brought into contact with and ligate tissue of the blood vessel upon compression of the clip around the blood vessel, whereby the clip is adapted to both cut through and ligate the blood vessel upon compression of the clip around the blood vessel.
